(19) United States
(12) Patent Application Publication
(10) Pub. No.: US 2007/0005120 A1
(43) Pub. Date: Jan. 4, 2007

(54) Method of intracanalicular laser dacryocystorhinostomy

(75) Inventors: Fco. Javier Arcusa Villacampa, Sant Cugat del Valles (ES); Mailin Auxiliadora F. Lissott, Sant Cugat del Valles (ES); Felix J. Alanon Fernandez, (US); Miguel A. Alanon Fernandez, (US); Filiberto Rosado Carazo, Madrid (ES)

Correspondence Address: CROWELL & MORING LLP;INTELLECTUAL PROPERTY GROUP, P.O. BOX 14300, WASHINGTON, DC, 20044-4300, US

(73) Assignee: EQUIPSA S.A., Madrid (ES)

(21) Appl. No.: 11/169,759

(22) Filed: Jun. 30, 2005

(51) Int. Cl. A61N 5/06

(52) U.S. Cl. 607/89

(57) ABSTRACT

Method for performing transcanalicular dacryocystorhinostomy which comprises the steps of inserting of a suitable fiber optic which is connected to a solid state laser that emits a laser beam with a wavelength of about 700 nm to about 1600 nm through a lacrimal duct, using an endonasal endoscope to position an end of the fiber optic to a location where perforation is to be formed, firing the laser until perforation to form an osteotomy, and optionally enlarging of the osteotomy.

### FIELD OF THE INVENTION

The present invention relates to a surgical technique that forms an osteotomy from the internal part of the lacrimal sac, without any need for incisions in the skin, opening up a clean passage through the perforation made in the nose bone. This technique is used to treat patients suffering from an obstruction of the nasolacrimal channel and allow tear to pass through the incision.

### BACKGROUND OF THE INVENTION

Normally, tears are drained from the eye through the nasolacrimal drainage, or lacrimal, system, which includes an upper and lower canaliculus, each of which communicates with its respective eyelid through an opening known as the punctum. The other ends of the canaliculi join together to form the common canaliculus which then enters the lacrimal sac. The tears then drain through the lacrimal sac into the upper end of the nasolacrimal duct. The lower end of the nasolacrimal duct opens into the nose. The tears bathe the eye and then flow into the upper and lower punctum, which are located on the inner upper and lower lid margins. The tears then drain through the superior and inferior canaliculi, common canaliculus, lacrimal sac, and down the nasolacrimal duct into the nose.

The nasolacrimal duct can become obstructed either congenitally or as an acquired obstruction in adulthood. When the nasolacrimal duct becomes obstructed, tears can no longer drain from the surface of the eye through the lacrimal system into the nose. The tears therefore well up over the eyes and spill over the lids onto the face. The patient has to constantly dab the eyes with a tissue. In addition, tears stagnate in the lacrimal sac which allows bacteria to multiply. The lacrimal sac then becomes infected (dacryocystitis). Dacryocystitis causes the lacrimal sac to become swollen, red and painful. Pus exudes from the lacrimal sac through the canaliculi onto the eye. This results in purulent material constantly covering the eye. In time, the dacryocystitis does not respond to antibiotics and surgery becomes necessary.

A surgical technique known as dacryocystorhinostomy (DCR) is used to correct nasolacrimal duct obstruction. In a DCR, a new opening (ostium) is created between the lacrimal sac and the nose. This allows tears to flow from the lacrimal sac through the DCR ostium into the nose. The traditional technique, often referred to as an open or incisional DCR, requires an incision on the side of the nose. This surgical technique makes the osteotomy from a cutaneous incision in the canthus (zone between the nose and eye. A large DCR ostium is created by making a 17 mm or wider opening in mucosa and bone. An open DCR is generally performed under general anesthetic, its surgical time is much longer, has significant morbidity, a prolonged recovery, and the threat of scarring and hemorrhage.

A transnasal endoscopic DCR, in contrast, requires no incision, and has much less morbidity, no risk of scarring, and a quick recovery time. An endoscope DCR may be performed using lacrimal or sinus surgery instruments, a laser, or a balloon catheter. The endoscopic DCR ostium is about 5 to 9 mm. A stent or a tube is often used to keep the DCR ostium open after surgery to prevent its closure caused by postoperative inflammation and scarring.

The foundations of the procedure on the excretory lacrimal sac are the opening of the lacrimal sac, drainage at nose cavity and restoration of the lacrimal passage. The appearance of lasers and of the endoscope has permitted modification of the original surgical technique, establishing the bases for minimally invasive surgery of the lacrimal system. In 1992, Christenburry (1) described the transcanalicular approach for the first time. Compared to the classic approach, this surgical technique presents a series of advantages such as: less tissue trauma, absence of cutaneous scarring, no damage caused to the medial canthal tendon or angular vessels, no bone fracturing, minimal bleeding and intra and postoperative pain, shorter surgical time, postsurgical morbility very low and a quick return to usual activity (2).

This surgical technique, however, requires a long learning curve, and also a nasal endoscopy. An osseous opening is made from inside the nose, using a sharp instrument to make “bites,” crossing the mucosas, nasal bone and aiming to come out at the lacrimal sac.

U.S. Pat. No. 5,345,948, to O'Donnell, discloses a method of performing translactrimal laser dacryocystorhinostomy. The method utilizes a video endoscope that is inserted through a punctum and canaliculus, and the laser used is not an infrared laser. This method, however, have several disadvantages. For example, due to the diameter of the fiber optic, it is difficult to visualize the area in which the laser has to be positioned, therefore making the control and manipulation inside the lacrimal sac very difficult. In addition, it is generally very difficult to conduct a bimanual operation in a narrow place. Furthermore, the maximum diameter of the laser probe used is about 200 μm, requiring at a long time for the laser to perforate and vaporize the tissues, resulting in a prolonged surgery procedure.

There is therefore a need for an improved DCR method that avoids the shortcomings of the prior art techniques.

### SUMMARY OF THE INVENTION

Disclosed herein is a method for intracanalicular dacryocystorhinostomy performed using a solid state laser, for example a 980 nm diode laser, with endonasal endoscopy vision (not an intracanalicular endoscope). This is a novel surgical technique which allows perforation of the nose bone (osteotomy) from the lacrimal sac by inserting an optic fiber, for example a 600 micrometer laser fiber, into the lacrimal canaliculus, without any need for incisions in the skin. The surgical process is carried out under local anesthetic, in a short period of time, without causing external scarring. Preferably, the solid state laser has emits a wavelength in the infrared spectrum from about 700 nm to 1,600 nm. The visualization of the red spot of the laser is done by endonasal transillumination with the endoscope.

The present method employs an endonasal endoscope. This allows the surgeon a wider view and higher resolution of the image obtained than if visualization is done by video endoscope inserted through a punctum and canaliculus. For example, the fiber optic inserted through a canaliculus is about 0.5 mm while a nasal endoscope uses a fiber optic with a diameter of about 2.7 mm or more. With this improved visualization, the laser fiber optic can be easily and efficiently directed to the desired location and osteotomy can be created more efficiently. Enlargement, if necessary of the osteotomy, visualization and future examination of the permeability of the new passage can also be done more easily according to the present invention.

In one alternative embodiment, the present invention provides a method of intracanalicular dacryocystorhinostomy where osteotomy is enlarged by introducing a second laser fiber from the nose. An endonasal endoscope greatly facilitates this operation and shortens patient recovery time.

In one embodiment, the method of transcanalicular dacryocystorhinostomy according to the present invention comprises the steps of inserting of a suitable fiber optic which is connected to a solid state laser through a lacrimal duct, using an endonasal endoscope to position an end of the fiber optic to a location where perforation is to be formed, firing the laser until perforation to form an osteotomy, and optionally enlarging of the osteotomy.

Preferably, the laser emits a laser beam with a wave length of about 700 nm to about 1600 nm. Preferably, the wavelength is 980 nm. A suitable laser for the invention has a power of about 1 to 50 W, and a mode of operation which may be continuous wave or pulse.

In one embodiment, the fiber optic has a diameter ranging from about 100 μm to about 1000 μm, preferably 400 μm or 600 μm.

According to another embodiment, the laser further emits a pilot beam which is viewed through the endonasal endoscope for properly positioning the end of the fiber optic.

The method of the present invention can be performed under local anesthesia.

The osteotomy formed according to the present invention may be further enlarged, either from the lacrimal sac, or from inside the nose with another suitable laser.

Bicanalicular intubation is preferably performed following the formation of the osteotomy, for example with a silicone tube covered with PVP and prolene filament assisted by a probe.

In a preferred embodiment, the method of the present invention utilizes a powerful laser coupled to a large diameter fiber optic to reduce the time of surgery. For example with a fiber optic of 600 μm, only a third of time is needed if a fiber optic having a diameter of 200 μm is used with a comparable laser.

### BRIEF DESCRIPTION OF THE DRAWINGS

FIGS. 1-6 graphically describe the steps of an embodiment of the DCT-TC technique of the present invention.

FIG. 7 shows canalization with the fiber of the laser of the excretory lacrimal duct through the superior punctum.

FIG. 8 shows the pilot beam light during laser activation and entrance of the laser fiber in the nasal cavity (middle meatus) through the sac mucosa.

FIG. 9 shows that the new ostium is enlarged in an anteroinferior direction in relation to the initial opening.

FIG. 10 shows an endonasal endoscopic view of the drainage of purulent material following the opening of the osteotomy at middle meatus.

FIG. 11 is an endonasal endoscopic image showing absence of bleeding following intubation of new tract.

FIG. 12 is an endonasal endoscopic view showing fluorescein staining of the new ostium, by Jones Test I with direct videoendoscopic control 1 year after surgery.

### DETAILED DESCRIPTION OF THE INVENTION

The present invention provides a method for carrying out transcanalicular dacryocystorhinostomies using a diode laser (DCR-TC). Specifically, the DCR-TC method of the present invention is performed using a solid state laser with wave length in the infrared spectrum of about 700 nm to about 1,600 nm, with endonasal endoscopy vision. This is a novel surgical technique which allows perforation of the nose bone (osteotomy) from the lacrimal sac by inserting an optic fiber into the lacrimal canaliculus, without any need for incisions in the skin. The surgical process is carried out under local anesthetic, in a short period of time, without causing external scarring.

The use of a solid state laser allows the method of the present invention to achieve its desired results. Specifically, it was surprisingly discovered that the use of solid state laser with a wave length in infrared spectrum from about 700 nm to about 1,600 nm allows instantaneous cauterization and prevents bleeding from occurring. As discussed above, an endonasal endoscopic view allows easy control of the laser fiber optic and the formation and enlargement of the osteotomy (e.g. by controlling the size of the osteotomy). It also allows close examination of the anatomy of the nose and resolve problems or potential complications that may prevent the procedure from succeeding. It further allows enlargement the osteotomy from the nose.

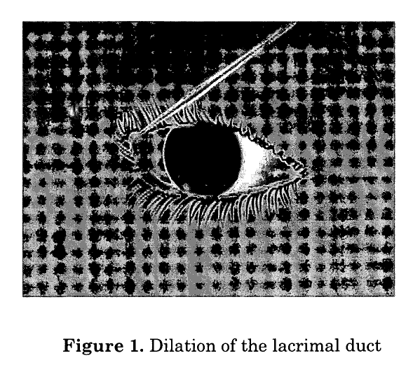

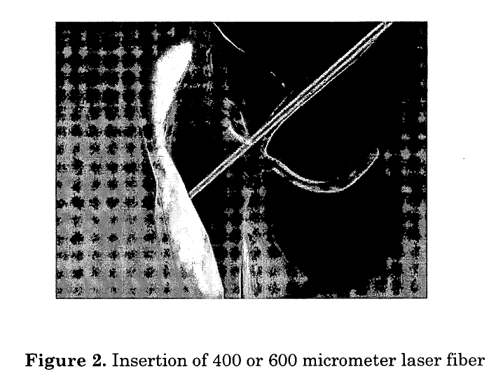

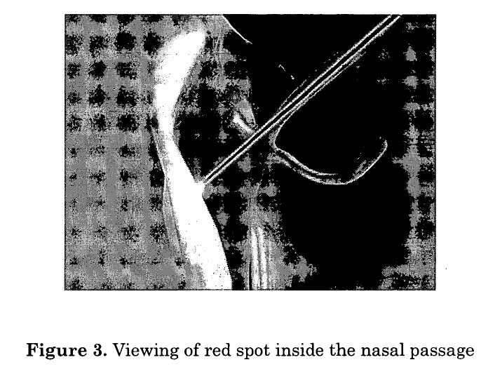

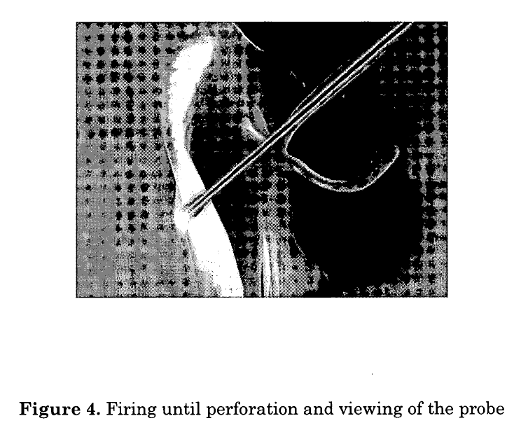

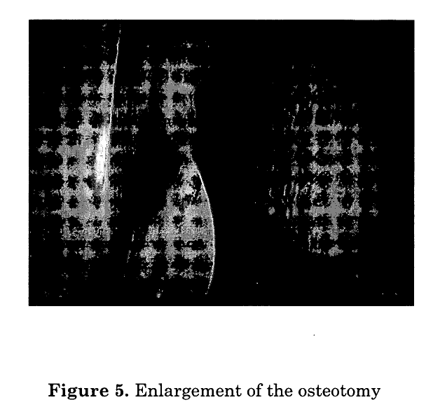

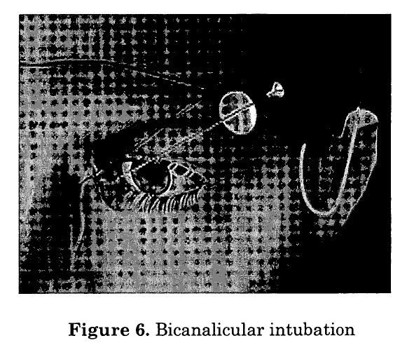

As show in FIGS. 1-6, the method of the present invention comprises the steps of dilation of a lacrimal duct; insertion of a suitable laser fiber; positioning and viewing with an endonasal endoscope of the positioning of the laser end where perforation is to be formed (e.g. via a pilot beam projected from the laser fiber inside the nasal passage); when the positioning is determined to be proper, firing of the solid state infrared laser until perforation and examination of the osteotomy, enlargement of the osteotomy with the laser, and bicanalicular intubation with a suitable bicanalicular stent, for example the Ritleng Bicanaliculus Intubation system from FCI Ophthalmics, Inc., Marshfield Hills, Mass. 02051.

It is to be recognized that insertion of the laser fiber may be via either the inferior or superior canaliculus. The endonasal endoscope is inserted through the nose to visualize the pilot beam.

One of ordinary skills will recognize that with suitable devices the above steps can be performed with techniques well-known in the art. In a preferred embodiment, the method of the present invention comprises the steps of: dilating a lacrimal duct with a suitable dilator; passing a bone cutting solid state laser through the punctum; passing the bone cutting laser into the lacrimal sac; visualizing the laser through an endonasal video endoscope; positioning the laser towards an medium turbinae bone; activating the laser; and forming a full-thickness fistula through said lacrimal sac and through the medium turbinate bone.

The method of the present invention operates from the canaliculus and the lacrimal sac towards the inside of the nose by the insertion of a laser fiber. It is therefore non-invasive and requires no incisions in the skin, which completely avoids any scarring. In addition, surgery according to the present invention can be performed under local anesthesia, and the technique is easy to mater by a surgeon who wishes to learn the technique.

Enlargement of the osteotomy according to the instantly disclosed method may not only be from the lacrimal sac, but also from inside the nose with a laser fiber.

In a preferred embodiment, an solid state laser is used for the method of the present invention. This laser emits a wavelength of about 700 to 1600 nm, has a power of about 1 to 50 W. Its mode of operation is CW (continuous wave) or pulse mode. It has a pilot beam for laser positioning. It uses fiber optic with a diameter ranging from about 100 μm to about 1000 μm.

The method of the present invention preferably uses a nasal endoscope, thereby avoiding the need of using the other lacrimal punctum for endoscopic viewing and positioning of the laser spot. Due to the importance of the lacrimal punctum and inferior canaliculus in tear drainage, the surgeon should act as little as possible on them for surgical maneuvers, using the lacrimal punctum and the superior canaliculus for canalizing the tract with optical fiber.

Chief among the advantages of the transcanalicular technique is the fact the energy is transmitted by the orbit lacrimal anatomic tracts to a natural cavity such as the nasal passage, therefore not endangering the eye or periocular structures. Furthermore the technique recalls nasolacrimal probing and a trained ophthalmologist is familiar with its tactile sensation. (5).

The laser energy is transmitted by an optical fibre, the precise ablation being in the area marked by its terminal. The method of the present invention performs the incision, excision, vaporization and coagulation of the tissues at the same time.

While not willing to be bound by any theory, it is believed that the surgical action of the solid state laser used according to the present invention is advantageous because of the absorption of its energy by the target tissue and its transformation into heat (thermal effect) (6), capable of destroying mucous tissue and lacrimal bone wall, creating a fistula between the lacrimal sac and nasal passage (5).

An optical fiber with a small diameter enables a more effective cut to be obtained for a given power in comparison to fibers with larger diameters, as the energy/ surface area ration (energy density) is greater the field of radiation is narrower and more concentrated (6).

For the osteotomy, pulses were used with an emission time/pause for the ablation of a portion of the tissue with each impact and to obtain a relaxation of the tissue temperature through the pause. This way, an accumulative thermal effect is avoided, diminishing carbonization and achieving a more precise cut (6).

The coagulant effect is owing to the wavelength of the solid state laser used which ensures good absorption by the oxyhemoglobin, on account of which it may be used in patients with blood dyscrasia.

Silicon probes with prolene filament are preferably used to minimize traumatism to the nasal mucosa. In comparison, intubation with metal guides may cause lacerations in this area, pain during the surgical operation and synechia in the postoperative period.

Polyvinylpyrrolidone covering has been associated with higher patient tolerance, better tear drainage by capillarity and a lower incidence of inflammatory reaction, causes of canalicular obstruction and failure of the DCR (7).

A preferred location for the osteotomy and the opening to nasal cavity is the portion before the middle nasal concha, which can be marked through transillumination with the PILOT beam of light that is produced in the optical fiber when the solid state laser is activated (6). This location should help avoid cases of epiphora with irrigation of the permeable tract, perhaps due to a localization of the superior and posterior osteotomy with respect to the desirable place (drain syndrome) (8-9).

### EXAMPLES

### Example 1

Summary: Forty-three transcanalicular DCR were performed using a 980 nm diode laser (Varius, Intermedic, Barcelona, Spain) and bicanalicular intubation with silicone tube and prolene filament, both PVP-covered (PVP Ritleng lacrimal intubation set, FCI, Issy-les-Moulineaux Cedex, France). The results were analysed using a prospective, interventional, non randomized and non comparative study. Local and topical anaesthesia were used in patients with a clinical history of epiphora or dacryocystitis for nasolacrimal obstruction. A diode laser was used to effect a vaporization of the lacrimal sac, osteotomy and vaporization with coagulation of nasal mucosa. The mean duration of surgery was 14 minutes (range 7 to 29 minutes). In all cases, and during a two-month period, bicanalicular intubation was carried out using a silicone tube and prolene filament. Follow-up was between 4 to 38 months. The degree of epiphora was evaluated using the Munk scale, and lacrimal permeability was evaluated using Jones I and II tests by direct videoendoscopic control in all cases.

Patient ages ranged between 27 and 82 years, the mean being 59.1 years. 86% were female and 14% were male, 16 were undergoing the procedure on the right side and 27 the left. Two patients (4.65%) had previously undergone an external dacryocystorhinostomy. Six patients (13.95%) had a personal history of acute dacryocystitis and 4 (9.30%) of chronic dacryocystitis. The remaining 33 cases (76.74%) were due to idiopathetic obstruction symptomatic at distal level. It was not necessary to modify the anticoagulant treatment being followed by one of the patients (2.32%) prior to surgery.

Of the 43 DCR-TC operations, 39 cases are asymptomatic (90.7%), 2 (4.65%) had epiphora (degree 2 on the Munk scale) and permeable tract, 1 case (2.32%) presented lower canaliculi obstruction, and 1 patient showed total osteotomy closing.

Material and Methods

All patients were examined by both the ophthalmologist and otolaryngologist, including an examination by slit lamp, a palpebral evaluation and an examination of nasal passages with a 30° endoscope 4 mm in diameter (Storz, Tuttlingen, Germany). Nasolacrimal duct obstruction was diagnosed using the Jones I and II tests with direct videoendoscopic control. Furthermore, prior digital dacryocystography was carried out on patients with a clean reflux to the irrigation by contralateral point to establish the level of the obstruction.

The surgery was carried out in an outpatient surgery with local and topical anesthetic. The nasociliary region was infiltrated and infraorbital and infratrochlear nerve blocks were carried out with 1 ml in each zone of a mix of 1.5 ml of Mepivacaine at 2% and 1.5 ml of Bupivacaine at 0.75%, the anesthetic of the nasal passages being topical via a cotton swab soaked in Tetracaine at 1% and Epinephrine 1/100,000 positioned 15 minutes before surgery.

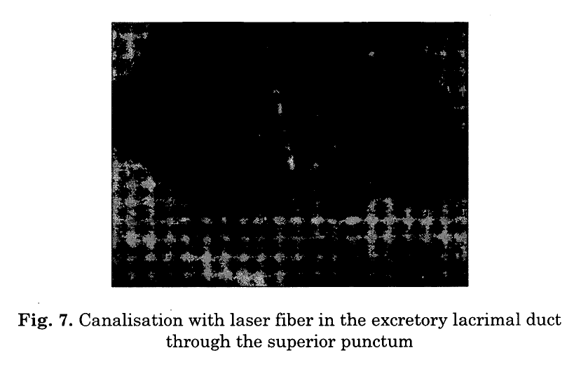

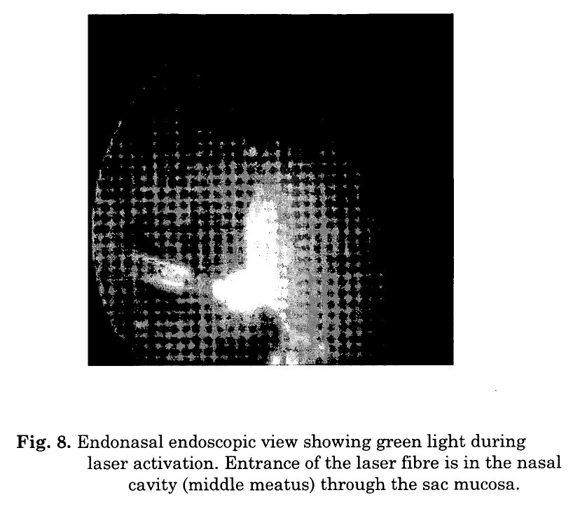

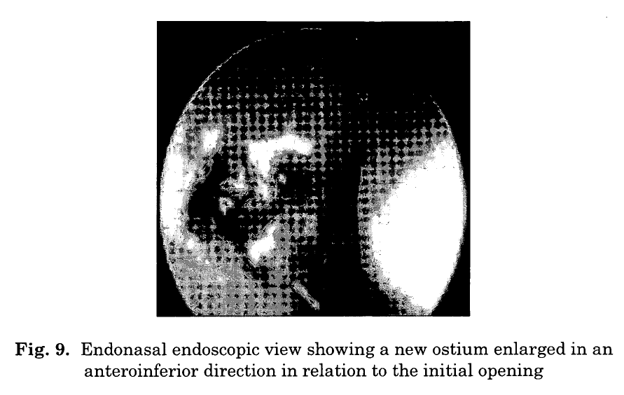

After dilating the superior lacrimal punctum, a 400 micrometer optical fiber was inserted into it (FIG. 7) (Intermedic, Barcelona, Spain) and following the natural anatomic tracts, through the superior canaliculus superior, common canaliculus and lacrimal sac until coming in contact with the medial wall of the lacrimal sac, confirmed by the hardness of the lacrimal bone (3, 4). At this moment, pulses of energy were fired with the diode laser, a mean of 15 pulses (range 10-40 pulses), with a duration of 50 ms and pauses of 50 ms and a power ranging from 8 and 20 watts until the fiber passed to the nasal passage (FIG. 8), mean energy 6 joules (range 4-30 joules). Immediately afterwards, assisted by direct viewing via endoscope, the initial osteotomy, by transcanalicular via, was amplified in an anteroinferior direction (2.5) through the juxtaposition of confluent impacts following the shape of a circle (FIG. 9) until obtaining a window of some 8-10 mm in diameter.

Finally, bicanalicular intubation was carried out with silicone tubes covered with PVP and prolene filament assisted by the Ritleng probe (FCI, Issy-les-Moulineaux Cedex, France).

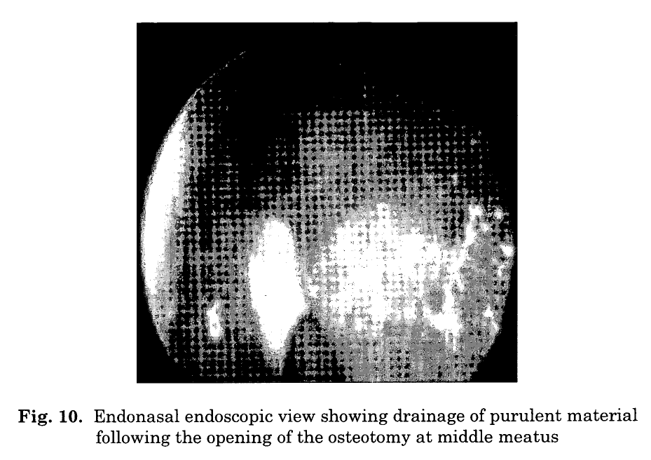

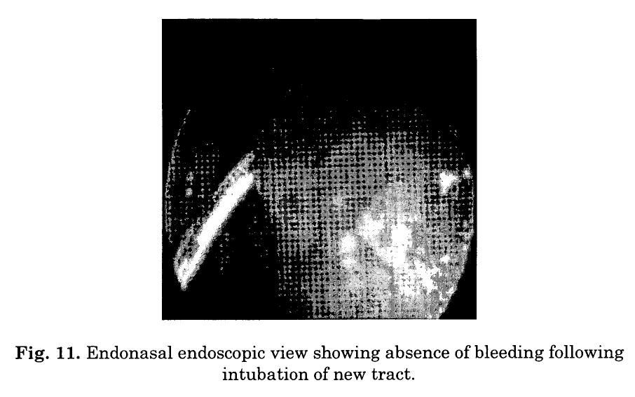

The position and size of the osteotomy as well as the collection of the silicone tubes (FIGS. 10 and 11) through the nasal cavity were controlled in real time by video endoscope.

The mean duration of surgery was 14 minutes (range 7-25 minutes).

In the postoperative treatment, antibiotic-corticoid drops were instilled 4 times a day over 1 week and an antibiotic-corticoid-vasoconstrictor nasal spray inhaled 3 times a day over 5 days.

Nasal endoscopy was performed 1 month, 2 months and 4 months after surgery and subsequent check-ups were carried out on a six-monthly basis.

The interposition material was removed 2 months after the surgical procedure.

The study is prospective. The same surgeons who performed surgery followed up on the same patients in every case.

Results

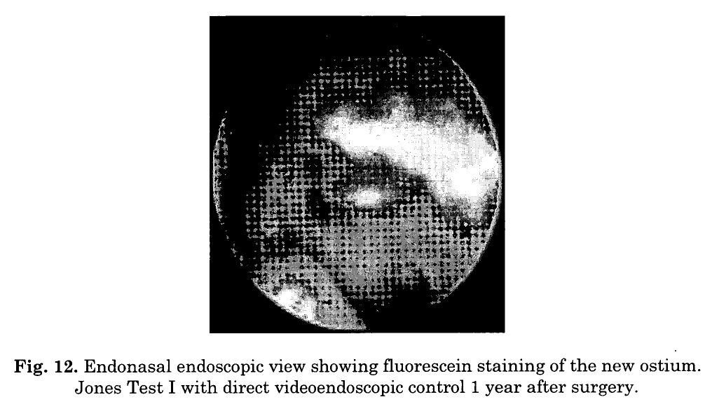

The surgical procedure was considered to be successful if the epiphora disappeared and in the Jones I test the fluorescein staining of new surgical ostium was checked directly by endoscope (FIG. 12) 4 months after surgery (2 months after removal of the silicone tubes).

Of a total of forty-three patients, thirty-nine displayed absence of epiphora with Jones test I positive.

Two patients displayed occasional epiphora and mucous secretion with permeable irrigation with Jones test I negative and Jones test II with positive direct videoendoscopic control and one obstruction of the inferior canaliculus by iatrogenic fibrosis 2 months after removing the intubation, these cases corresponding to patients with idiopathetic nasolacrimal duct obstruction. In these three cases residual epiphora was objectified in the face of external aggression (air, cold, smoke . . . ) and the need for drying between 2 and 24 times a day (grade 2 on the Munk scale).

A patient with a history of acute dacryocystitis displayed a complete closing of the new ostium.

In one case, the new tract could not be intubed due to poor patient collaboration, remaining permeable in the postoperative checks.

As a post-surgical complication, a patient, in whom 20 W pulses of power were used, displayed edema and palpebral erythema in the internal canthal region 72 hours after surgery but were resolved without requiring treatment.

In a patient, the bicanalicular intubation was removed 20 days after surgery following the extrusion of the knot via the nasal passage due to sneezing. The tract remained permeable in the post-surgical checks.

One case displayed laceration of the lacrimal puncta and another nasal epithelialization of the silicone tubes, corresponding to our first cases, where an attempt was made at maintaining the intubation of the new tract over more than two months.

The mean postoperative follow-up was 20.3 months (range 4-38 months).

All patients returned to their normal activity after 48 hours.

In conclusion, transcanalicular dacryocystorhinostomy carried out using a diode laser is a useful method because it does not cause cutaneous scarring, it hardly produces pain and bleeding, it respects the lacrimal pump, it needs less surgical time, it can be carried out in an out-patient surgery and it generates minimal intra and post-surgical morbidity

### BIBLIOGRAPHY

1. Christenburry J D. Translacrimal laser dacryocystorhinostomy. Arch Ophthalmol 1992; 110: 170-171.
2. Piaton J M, Keller P, Limon S, Quenot S. First line endonasal dacryocystorhinostomy Technique and results. Comparison between diode laser and electrocautery instrument. Study based on 422 procedures. J Fr Ophtalmol 2002; 25: 135-145.
3. Toledano Fernández N. Dacriocistorrinostomía transcanalicular con láser. En: Toledano Fernández N. Manejo actual de las obstrucciones del conducto nasolagrimal. Comunicación Solicitada 77 Congreso de la Sociedad Española de Oftalmología. Barcelona: Ed. Sociedad Española de Oftalmología 2001; 107.
4. Pérez Santoja J J, Alió Sanz J L, Ismail M M. Dacriocistorrinostomía endocanalicular con láser versus dacriocistorrinostomía endoscópica con láser. Arch Soc Esp Oftalmol 1997; 72: 309-312.
5. Levin P S, Stormo Gipson D J. Endocanalicular láser-assisted dacryocystorhinostomy. An anatomic study. Arch Ophthalmol 1992; 110: 1488-1490.
6. Larizgoitia I. El Láser. Agéncia d'Avalució Tecnologia Recerca Médiques. http://www.aatm.es.
7. Toledano Fernández N. Garcia Sandoval B, Beneito Pérez P, Grande Baos C. Tratamiento quirúrgico de las obstrucciones del conducto nasolagrimal. En: Toledano Fernández N. Manejo actual de las obstrucciones del conducto nasolagrimal. Comunicación Solicitada 77 Congreso de la Sociedad Española de Oftalmología. Barcelona: Ed. Sociedad Española de Oftalmología 2001; 71-83.
8. Migliori M E. Endoscopic evaluation and management of the lacrimal sump syndrome. Ophthalmic Plast Reconstr Surg, 1997; 13: 281-284.
9. Garcia Sandoval B, Toledano Fernádndez N. Plaza Mayor G. Dacriocistorrinostomía Endoscópica Intranasal. En: Toledano Fernández N. Manejo actual de las obstrucciones del conducto nasolagrimal. Comunicación Solicitada 77 Congreso de la Sociedad Española de Oftalmología. Barcelona: 2001; 85-91.

We claim:

1. A method transcanalicular dacryocystorhinostomy comprising the steps of inserting of a suitable fiber optic which is connected to a solid state laser through a lacrimal duct, using an endonasal endoscope to position an end of the fiber optic to a location where perforation is to be formed, firing the laser until perforation to form an osteotomy, and optionally enlarging of the osteotomy.

2. The method according to claim 1, wherein the laser emits a laser beam with a wave length of about 700 nm to about 1600 nm.

3. The method according to claim 2, wherein the laser has a power of about 1 to 50 W, and a mode of operation which is continuous wave or pulse.

4. The method according to claim 2, wherein the fiber optic has a diameter ranging from about 100 μm to about 1000 μm.

5. The method according to claim 2, wherein the laser further emits a pilot beam.

6. The method of according to claim 1, wherein the method is performed under local anesthesia.

7. The method according to claim 1, wherein enlargement of the osteotomy is from the lacrimal sac, or from inside the nose.

8. The method according to claim 7, wherein the enlargement of the osteotomy is effected with a fiber optic connected to a solid state laser emitting an infrared laser beam having a wavelength of about 700 nm to about 1600 nm.

9. The method according to claim 2, wherein the wavelength of the laser beam is about 980 nm.

10. The method according to claim 4, wherein the fiber optic has a diameter of about 600 μm.

11. The method according to claim 1, wherein the method further comprises performing bicanalicular intubation.

12. The method according to claim 11, wherein bicanalicular intubation is performed with a silicone tube covered with PVP and prolene filament assisted by a probe.
